(19) United States
(12) Patent Application Publication
(10) Pub. No.: US 2009/0234278 A1
(43) Pub. Date: Sep. 17, 2009

(54) CATHETER INSERTION SHEATH WITH ADJUSTABLE FLEXIBILITY

(75) Inventor: Kai Eck, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/301,112

(22) PCT Filed: Apr. 10, 2007
(86) PCT No.: PCT/IB2007/051284

§ 371 Date: Nov. 17, 2008

Related U.S. Application Data
(60) Provisional application No. 60/747,822, filed on May 22, 2006.

(51) Int. Cl. A61M 25/092

(52) U.S. Cl. 604/95.01

(57) ABSTRACT

The present invention includes a sheath (10) for guiding materials in a body cavity. The sheath comprises a tubular structure having an exterior surface (12) of a sidewall (13) and a lumen (14) enclosed by an interior surface (16) of the sidewall. The sidewall has a duct (18) containing a magnetorheological fluid.

Also presented is a method for navigating a sheath (60) comprising introducing the distal end of the sheath to a passage (62) in the patient's body; manipulating the rigidity of the magnetorheological fluid by applying a magnetic field; and positioning the sheath. A navigable catheter and sheath assembly is also presented.

### FIELD OF THE INVENTION

This invention relates to sheaths for use with catheters and other applications. Specifically, the invention relates to flexible sheaths with variable rigidity.

### BACKGROUND OF THE INVENTION

Catheters are used extensively in the medical field in various types of procedures, including invasive procedures. Minimally invasive surgery involves operating through small incisions, through which instruments are inserted. These incisions are typically 5 mm to 10 mm in length. Minimally invasive surgery is typically less traumatic than conventional surgery, due in part to the significant reduction in incision size. Furthermore, hospitalization is reduced and recovery periods are shortened as compared with conventional surgery techniques. Catheters may be tailored to a particular size or form, depending on the incision and the size of the body cavity or vessel.

The steering of catheters inside the body is a challenging and time-consuming task in many applications, such as angioplasty and electrophysiological interventions. To avoid extended exposure of the physician to radiation, remote control operation systems are under development. One difficulty with remotely controlled catheters involves transmitting forces from the back end of the catheter to the tip. A catheter that is too flexible is unable to transfer force, whereas a catheter that is too stiff is unable to maneuver through the difficult curvatures.

### SUMMARY OF THE INVENTION

The present invention includes a sheath (10) for guiding materials in a body cavity. The sheath comprises a tubular structure having an exterior surface (12) of a sidewall (13) and a lumen (14) enclosed by an interior surface (16) of the sidewall. The sidewall has a duct (18) containing a magnetorheological fluid.

Also presented is a method for navigating a sheath (50) adapted to guide materials in a patient's body, wherein the sheath has a distal end, a proximal end, and a sidewall having a duct (18) containing a magnetorheological fluid. The method comprises: introducing the distal end of the sheath to a passage (62) in the patient's body; manipulating the rigidity of the magnetorheological fluid by applying a magnetic field; and positioning the sheath. A navigable catheter and sheath assembly is also presented. The assembly comprises: a sheath (60) for positioning a catheter (64), and the sheath comprises a tubular structure having an a sidewall and a lumen enclosed by an interior surface of the sidewall. The sidewall has a duct containing a magnetorheological fluid. The assembly further comprises a catheter (64) adapted for insertion through the lumen of the sheath; a magnetic field generating apparatus (66) adapted to generate a magnetic field which manipulates the rigidity of the magnetorheological fluid.

### BRIEF DESCRIPTION OF THE DRAWINGS

FIG. 1 is a schematic of a catheter sheath with a U-shaped duct of magnetorheological fluid on the exterior sidewall in accordance with one embodiment of the invention.

FIG. 2 is a schematic of a catheter sheath with a W-shaped duct of magnetorheological fluid on the exterior sidewall in accordance with one embodiment of the invention.

FIG. 3 is a schematic of a catheter sheath with a duct of magnetorheological fluid circumscribing the exterior sidewall in accordance with one embodiment of the invention.

FIG. 4 is a schematic of a catheter sheath with multiple parallel ducts of magnetorheological fluid on the exterior sidewall in accordance with one embodiment of the invention.

FIG. 5 is a flow chart that schematically illustrates a method for navigating a catheter sheath in accordance with one embodiment of the invention.

FIG. 6 is a schematic of a catheter sheath and catheter assembly in accordance with one embodiment of the invention.

### DETAILED DESCRIPTION OF THE INVENTION

The invention describes a remote controlled sheath for insertion of catheters, or other materials. The flexibility or stiffness of the sheath can be controlled externally by modulating the strength of an applied magnetic field. The facile adjustment of the flexibility of the sheath provides the operator greater control and reduces the danger of causing damage to the patient tissue during catheter insertion. The sheath varies in rigidity because it contains a magnetorheological fluid that transitions between a rigid, solid-like state and a liquid fluid state as a function of magnetic field.

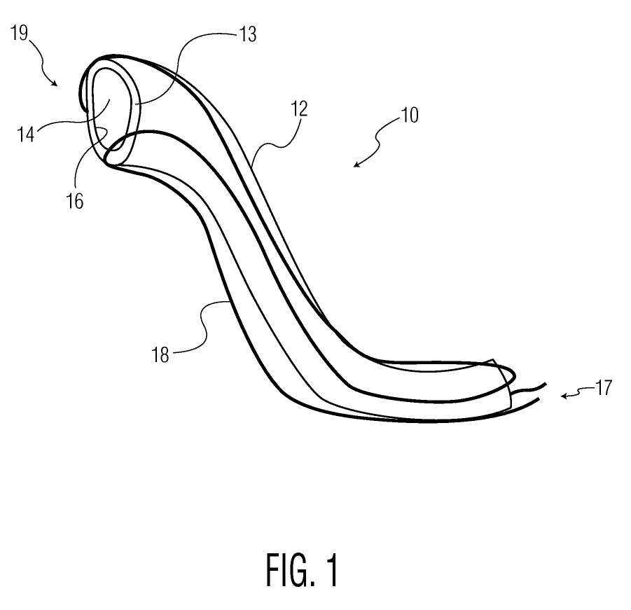

Referring to FIG. 1, a sheath 10 for positioning a catheter is shown as a tubular structure having an exterior surface 12 of a sidewall 13 and a lumen 14 enclosed by an interior surface 16 of the sidewall 13, the sidewall having a duct 18 containing a magnetorheological fluid. The lumen can be adapted to transport and position a catheter. The sheath is appropriate to transport and position catheters for a variety of purposes, including electrophysiology procedures, angioplasty, and ablation. The lumen can also be adapted to transport and apply coils, liquids, or other materials as appropriate.

The sheath 10 can be formed of a conventional, bendable tubing material of low stiffness, combined with a magnetorheological fluid (MRF) contained in a duct 18 on the sheath. When magnetic fields are applied, the MRF becomes rigid in regions exposed to local magnetic fields. As the strength of the magnetic field increases, the rigidity of the fluid increases. For applying such fields, an external magnetic coil can be employed. Alternatively, the magnetic field can be applied to the end of the sheath. With the magnetic field applied to one end of the sheath, the MRF itself acts as a line of high magnetical conductivity and causes the particles in the magnetorheological suspension to coagulate.

A magnetorheological fluid is a liquid that hardens near a magnetic field, and becomes liquid again when the magnetic field is removed. The term magnetorheological fluid (MRF) refers to liquids that solidify in the presence of a magnetic field. Magnetorheological fluids have micrometre scale magnetic particles, and the magnetorheological effect in fluids develops when the particle size is about 10 nanometers or larger. The particles can be iron, magnetite, cobalt, or other magnetic materials, and the surrounding liquid can be an oil, water, wax, or other solvent. Surfactants can be used to make the suspension more stable, for example, trapping particles in micelles to maintain separation.

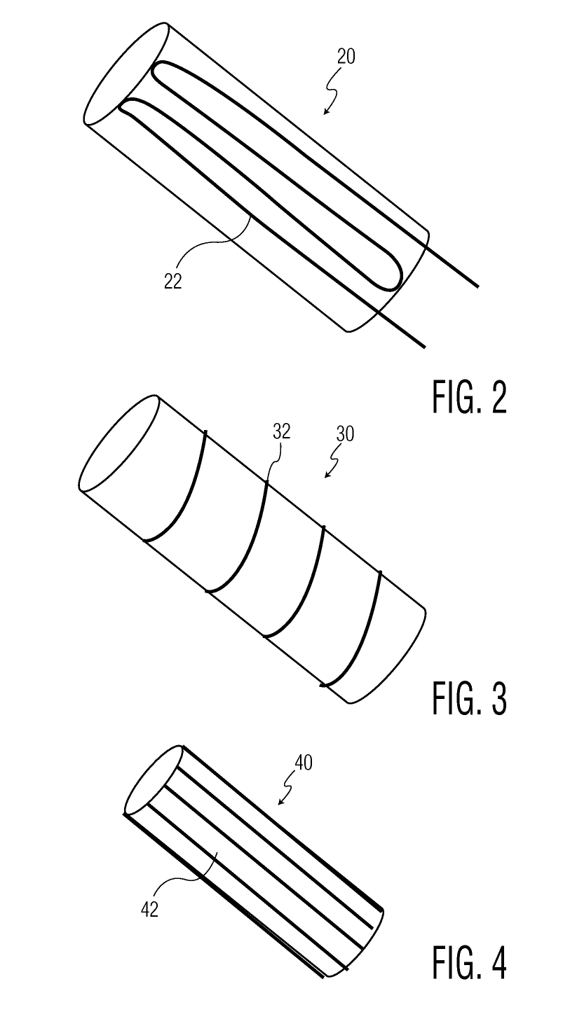

Again referring to FIG. 1, the duct 18 on the sheath 10 may extend from the proximal end 17 of the tubular structure to the distal end 19 of the tubular structure. The duct of the sheath can take a variety of configurations to optimize performance for various catheter insertion operations. For example, the duct may extend from the proximal end to the distal end of the tubular structure repeatedly, as shown in FIGS. 1 and 2.

FIG. 2 is a simplified schematic of a sheath 20, which is similar to the sheath 10 shown in FIG. 1. In FIG. 2, the duct 22 repeatedly extends between the distal and proximal ends of the sheath. In another embodiment of the invention, a serpentine pattern may continue around the full circumference.

Another exemplary pattern for the duct of MRF is shown in FIG. 3. Here, the duct 32 extends around the circumference of the sheath 30. The duct may be formed as a continual coil that wraps around the sheath, or alternatively may be formed from parallel concentric rings around the sheath.

FIG. 4 illustrates yet another embodiment of the invention in which the duct 42 is formed from several parallel segments running along the sheath 40 oriented substantially parallel to the sheath's longitudinal axis. In any of the configurations presented, the duct can reside on the exterior surface of the sheath sidewall, on the interior surface, or imbedded within the sheath sidewall.

The invention also includes a method for navigating a sheath adapted to guide materials, such as a catheter in a patient's body. In this method, the sheath, which has a duct containing a magnetorheological fluid, is introduced into a passage in the patient's body. A passage includes a body cavity or blood vessel.

In navigating the sheath and catheter in the passage, the rigidity of the magnetorheological fluid can be manipulated to facilitate advancement of the sheath by applying a magnetic field. Manipulating the rigidity of the MRF facilitates insertion and placement of the sheath. In positioning the sheath, if the passage includes a very tight radius of curvature, the rigidity of the MRF can be adjusted to allow more flexibility and maneuverability. Where the passage presents an area that is difficult to traverse, the rigidity of the MRF can be increased through the application of a magnetic field to permit transference of force in maneuvering the sheath.

Accordingly, the navigating and positioning of the sheath can include applying a magnetic field to the sheath and varying the applied magnetic field. The magnetic field can be applied as an external magnetic field. Alternatively, the magnetic field can be applied to one end of the sheath and the magnetic particles in the MRF can be used to create an internal magnetic field. Also, magnetic fields of different strength may be applied to the distal end of the sheath from the proximal end of the sheath.

The magnetic field can be adjusted to manipulate the rigidity of the MRF to create different regions of rigidity in the sheath. For example, regions at the distal end of the sheath could be in a flexible state, while regions at the proximal end of the sheath remain rigid.

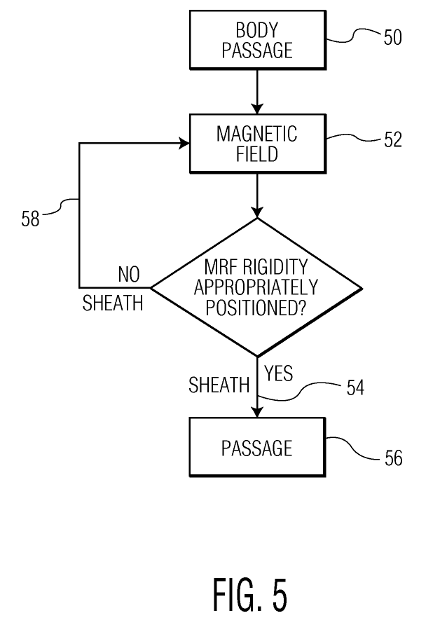

In navigating the sheath through the passage, the MRF may be controlled iteratively to correlate with conditions in the passage as the sheath advances by adjusting the applied magnetic field. Aspects of this process are illustrated in a flowchart in FIG. 5. The sheath is introduced to a body passage 50, and the rigidity of the MRF is manipulated via an applied magnetic field 52. If the MRF rigidity is appropriate to position the sheath 54, then the sheath is positioned in the passage as desired 56. Reference to positioning the sheath in the passage includes advancing the sheath, removing the sheath, and fixing the position of the sheath or catheter. If the MRF rigidity is not appropriate to position the sheath 58, then the rigidity of the MRF is manipulated by adjusting the magnetic field 52. This process can be repeated iteratively until the procedure is completed.

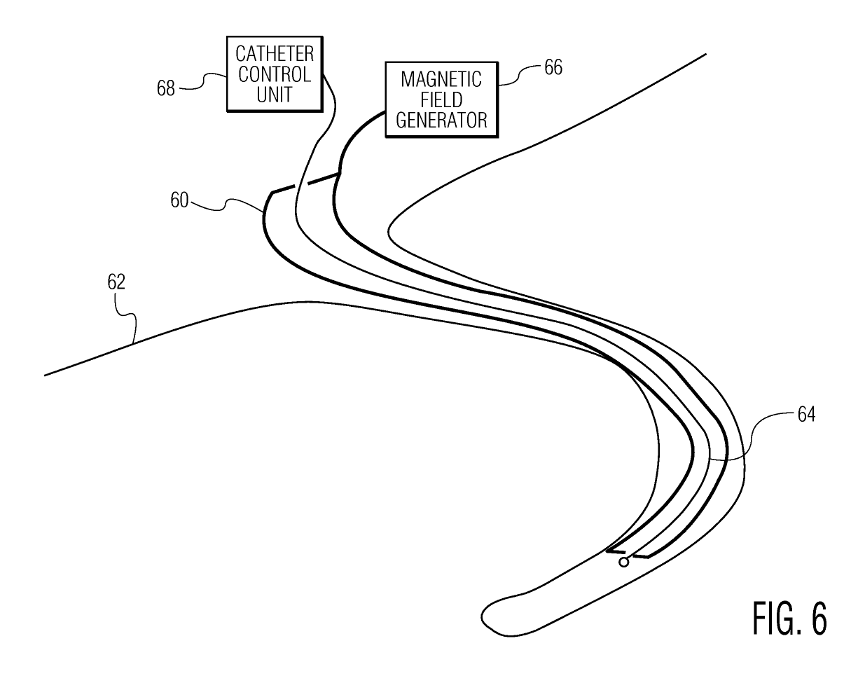

Another embodiment of the invention is a navigable catheter and sheath assembly. Referring to FIG. 6, the sheath 60 of the assembly is inserted into a body cavity or passage 62. The assembly includes a catheter 64 and a magnetic field generating apparatus 66 which is adapted to generate a magnetic field. The magnetic field serves to manipulate the rigidity of the magnetorheological fluid.

The assembly can also include a control unit 68 at the proximal end of the sheath. The control unit allows for controlling the sheath remotely. The control unit can be used to control the sheath, the catheter, or both.

The invention can be applied in the use of a multitude of catheters and sheaths for manipulations inside of the patient, with particularly useful applications in positioning electrophysiology (EP) catheters. Typical catheters may range in lengths of from about 35 cm to about 175 cm and more typically from about 50 cm to about 160 cm. The sheath will be approximately the same length.

The diameters of the catheter and sheath can vary between the distal and proximal ends. Preferably, the diameter should be as small as possible within the practical manufacturing limits so as to present the least trauma and the most conformability to the sheath. Typically, the distal portion of the sheath may vary with an outside diameter from about 0.6 mm (2 French) to about 6 mm (18 French) and more preferably, from about 0.6 mm (2 French) to about 2.3 mm (7 French). The outside diameter of the proximal portion can vary from about 1 mm (3 French) to about 6.3 mm (19 French) and more preferably, from about 1 mm (3 French) to about 2.7 mm (8 French). For example, the diameter of the distal portion may be 1.55 mm (4.5 French) and the diameter of the proximal portion may be 1.7 mm (5 French).

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A sheath (10) for guiding materials in a body cavity, the sheath comprising a tubular structure having an exterior surface (12) of a sidewall (13) and a lumen (14) enclosed by an interior surface (16) of the sidewall, the sidewall having a duct (18) containing a magnetorheological fluid.

2. The sheath of claim 1 wherein the duct (18) extends from a proximal end (17) of the tubular structure to a distal end (19) of the tubular structure.

3. The sheath of claim 2 wherein the duct (18) extends from a proximal end of the tubular structure to a distal end of the tubular structure repeatedly.

4. The sheath of claim 1 wherein the duct (18) resides on the exterior surface (12) of the sidewall (13).

5. The sheath of claim 1 wherein the duct (18) resides on the interior surface (16) of the sidewall (13).

6. The sheath of claim 1 wherein the duct (18) circumscribes the tubular structure.

7. The sheath of claim 1 wherein the duct (18) surrounds the tubular structure in a coil (32).

8. The sheath of claim 1 wherein the lumen (14) is adapted to transport and position a catheter.

9. The sheath of claim 1 wherein the magnetorheological fluid comprises magnetic particles having a particle size of 10 nanometers or greater.

10. The sheath of claim 1 further comprising a control unit (58) at the proximal end of the sheath.

11. A method for navigating a sheath (60) adapted to guide materials in a patient's body, wherein the sheath has a distal end, a proximal end, and a sidewall having a duct (18) containing a magnetorheological fluid, the method comprising:
introducing the distal end of the sheath to a passage (62) in the patient's body;
manipulating the rigidity of the magnetorheological fluid by applying a magnetic field; and
positioning the sheath.

12. The method of claim 11 wherein applying the magnetic field comprises varying an applied magnetic field.

13. The method of claim 11 wherein applying the magnetic field comprises applying a magnetic field to the distal end or the proximal end of the sheath (60).

14. The method of claim 11 wherein applying the magnetic field comprises applying differing magnetic fields to the distal end and the proximal end of the sheath (60).

15. The method of claim 11 wherein applying the magnetic field comprises adjusting an external magnetic field.

16. The method of claim 11 wherein manipulating the rigidity of the magnetorheological fluid creates differing regions of rigidity between the distal end and the proximal end of the sheath (60).

17. The method of claim 11 wherein navigating the sheath further comprises iteratively advancing the sheath through the passage and adjusting the applied magnetic field.

18. The method of claim 11 further comprising inserting a catheter transported in a lumen of the sheath.

19. A navigable catheter and sheath assembly comprising:
a sheath (60) for positioning a catheter (64), the sheath comprising a tubular structure having an a sidewall and a lumen enclosed by an interior surface of the sidewall, the sidewall having a duct containing a magnetorheological fluid;
a catheter (64) adapted for insertion through the lumen of the sheath; and
a magnetic field generating apparatus (66) adapted to generate a magnetic field which manipulates the rigidity of the magnetorheological fluid.

20. The navigable catheter assembly of claim 18 further comprising:
a control unit (68) at a proximal end of the sheath, wherein the sheath is remotely controlled by the control unit.
